(19) United States
(12) Patent Application Publication
(10) Pub. No.: US 2019/0284202 A1
(43) Pub. Date: Sep. 19, 2019

(54) PROCESS FOR THE MANUFACTURE OF DIAZEPINE DERIVATIVES

(71) Applicant: Hoffmann-La Roche Inc., Little Falls, NJ (US)

(72) Inventors: Fritz Bliss, Basel (CH); Xiaohua Du, Zhejiang (CN); Dawei He, Zhejiang (CN); Stefan Hildbrand, Basel (CH); Paul Spurr, Basel (CH); Wenfa Ye, Zhejiang (CN); Jianbing Zheng, Zhejiang (CN)

(73) Assignee: Hoffmann-La Roche Inc., Little Falls, NJ (US)

(21) Appl. No.: 16/434,438

(22) Filed: Jun. 7, 2019

Related U.S. Application Data
(63) Continuation of application No. PCT/EP2017/082729, filed on Dec. 14, 2017.

(30) Foreign Application Priority Data
Dec. 16, 2016 (CN) PCT/CN2016/110279

(51) Int. Cl. C07D 495/14

(52) U.S. Cl. CPC C07D 495/14

(57) ABSTRACT

The invention relates to a process for the manufacture of diazepine derivatives as defined in the description and in the claims.

### CROSS REFERENCE TO RELATED APPLICATIONS

This application is a continuation of International Application No. PCT/EP2017/082729, filed Dec. 14, 2017, claiming priority to Application No. PCT/CN2016/110279, filed Dec. 16, 2016, each of which are incorporated herein by reference in its entirety.

The invention relates to the manufacture of diazepine derivatives.

The invention relates in particular to a process for the manufacture of a compound of formula (I)

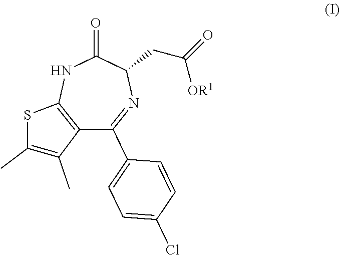

comprising the following steps:
(a) the reaction of a compound of formula (II)

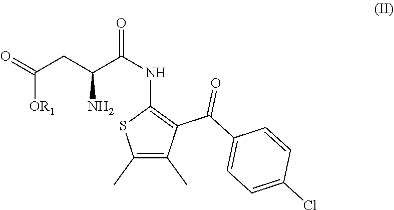

having an enantiomeric ratio of at least 70:30 with an acid to arrive at the compound of formula (I); and
(b) the crystallization of the compound of formula (I) obtained in step (a) from isopropyl acetate;
wherein R¹ is alkyl.
R¹ is advantageously tert.-butyl.

In step (a), the enantiomeric ratio of the compound of formula (II) as defined above can be for example between around 70:30 and around 100:0

The invention further relates to a process as defined above further comprising:
(c) the reaction of a compound of formula (I) as defined above with diethyl chlorophosphate, diphenyl chlorophosphate or bis(2-oxo-3-oxazolidinyl)phosphinic chloride and a base;
(d) the reaction of the product of step (c) with acetyl hydrazide followed by heating above room temperature to arrive at the compound of formula (I-d)

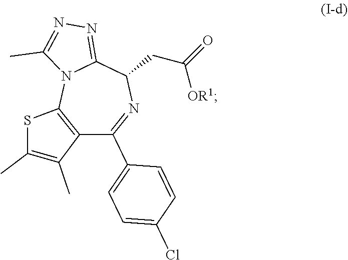

and
(e) the deprotection of the carboxyl group of the compound of formula (I-d) to arrive at the compound of formula (I-e)

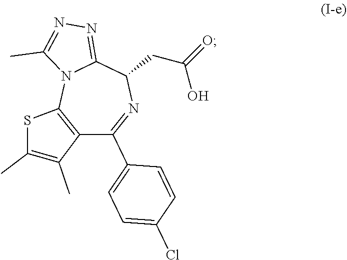

wherein R¹ is as defined above.

The compound of formula (I-e) is a useful building block for the synthesis of biologically active compounds (EP 0 989 131 B1; U.S. Pat. No. 5,712,274A, WO 2015/131113 A1, P. Filippakopoulos at al, Nature 2010, 468, 1067). The available processes for the manufacture of the compound of formula (I) are however not satisfactory (EP 0 989 131 B 1, Tetrahedron Letters 2015, 56, 3454-3457). In particular, racemization is encountered in several steps, making a classical resolution mandatory and thereby creating a yield loss. Furthermore, the known chiral resolution agent for the compound of formula (I-e) is cinchonidine, which is expensive and not readily available on technical scale and displays toxicology issues.

An efficient, high yielding process for the manufacture of the compound of formula (I-e) was therefore needed.

This problem was surprisingly solved by the process of the invention that provides the compound of formula (I), and hence subsequently the product of formula (I-e), in enantiomerically pure form.

The process according to the invention minimizes the loss of chiral information of expensive building block, enables the removal of any created undesired enantiomer by crystallization and hence avoids the classical resolution step with cinchonidine.

The invention allows a process for the manufacture of the compound of formula (I-e) having an enantiomeric ratio of at least 92:8 without chiral resolution during the entire process. The invention further provides a process for the manufacture of the compound of formula (I-e) in enantiomerically pure form, without chiral resolution, when the staring material (the compound of formula (I)) is enantiomerically pure. The invention thus provides a process for the manufacture of the compound of formula (I-e) having an enantiomeric ratio of between 92:8 and 100:0 without chiral resolution during the entire process.

It was surprisingly found that, in step (b), the racemic mixture of the compound of formula (I) remains in solution during the crystallization while the enantiomerically pure compound of formula (I) crystallizes and thus can be isolated by filtration. The compound of formula (I) can therefore be prepared in enantiomerically pure form even when the precursor compound of formula (II) possesses only a modest enantiomeric purity.

The invention thus also relates to a process for purifying the compound of formula (I) comprising the crystallization of the compound of formula (I) having an enantiomeric ratio of at least 70:30 from isopropyl acetate.

In the process of the invention, the purified compound of formula (I) crystallizes out of the solution. The racemate remains in solution and is removed into the mother liquor. The purified compound of formula (I) can be collected by filtration.

In the present description the term “alkyl”, alone or in combination, signifies a straight-chain or branched-chain alkyl group with 1 to 8 carbon atoms, particularly a straight or branched-chain alkyl group with 1 to 6 carbon atoms and more particularly a straight or branched-chain alkyl group with 1 to 4 carbon atoms. Examples of straight-chain and branched-chain C₁-C₈ alkyl groups are methyl, ethyl, propyl, isopropyl, butyl, isobutyl, tert.-butyl, the isomeric pentyls, the isomeric hexyls, the isomeric heptyls and the isomeric octyls, particularly methyl, ethyl, propyl, butyl and pentyl. A particular example of alkyl is tert.-butyl.

The term “peptide coupling agent” refers to a reagent used, for example in peptide coupling chemistry, to generate an active ester from a carboxylic acid. Examples of peptide coupling agents are DCC, DIC, EDC, BOP, PyBOP, PyAOP, PyBrOP, BOP-Cl, HATU, HBTU, HCTU, TATU, TBTU, HCTU, TOTU, COMU, TDBTU, TSTU, TNTU, TPTU, TSTU, TNTU, TPTU, DEPBT, CDI, as well as those mentioned below. The definitions of the above acronyms are well known to the skilled person.

The term “protecting group” signifies a group introduced into a molecule by chemical modification of a functional group to obtain chemoselectivity in a subsequent chemical reaction.

If one of the starting materials or compounds of formula (I) contain one or more functional groups which are not stable or are reactive under the reaction conditions of one or more reaction steps, appropriate protecting groups (as described e.g. in “Protective Groups in Organic Chemistry” by T. W. Greene and P. G. M. Wutts, 3rd Ed., 1999, Wiley, New York) can be introduced before the critical step applying methods well known in the art. Such protecting groups can be removed at a later stage of the synthesis using standard methods described in the literature.

The term “amine protecting group” or “amine protecting group” designates a protecting group of the amino group. Examples of amine protecting groups are 9-fluorenylmethyl carbamate (Fmoc), allyl carbamate (Alloc), vinyl carbamate (Voc), t-butyl carbamate (Boc), formamide, acetamide (or variously substituted acetamides such as chloroacetyl, trifluoroacetyl or phenylacetyl), arylamides, silyl, dibenzyl and variously substituted alkylsulphonamides. Fmoc is a particular protecting group of the amino group. Suitable amine protective groups and methods for their formation and cleavage are described in Protective Groups in Organic Chemistry, ed. J. F. W. McOmie, Plenum Press, 1973 and in T. W. Greene & P. G. M. Wuts, Protective Groups in Organic Synthesis, John Wiley & Sons, 3rd edition, 1999, and 2nd edition, 1991. A particular amine protecting group is Fmoc.

“Room temperature” can for example be around 20° C.

The following abbreviations are used in the present description. DCM=dichloromethane; EA=ethyl acetate; THF=tetrahydrofuran; DMF=dimethylformamide; t-Boc=tert-butyloxycarbonyl; Fmoc=9-fluorenylmethyloxycarbonyl.

The process according to the invention provides the first synthesis of the compound of formula (I) in enantiomerically pure form.

Water that is produced during the reaction of step (a) can be removed from the reaction mixture by distillation and the removed solvent/water mixture can be replaced by fresh solvent, in particular by isopropyl acetate.

The removal of water in step (a) can be done with a Dean Stark apparatus or by azeotropic distillation of the solvent and refilling with fresh solvent.

The removal of water advantageously forces the reaction of step (a) to go to completion.

The compound of formula (II) can be used at the begining of step (a) as a salt, for example a salt formed with an acid. In case a salt with an acid is used, step (a) is preceded by the treatment of the salt of the compound of formula (II) with a base, e.g. K₂CO₃, to arrive at the free base compound of formula (II).

The acid of step (a) can be for example acetic acid, formic acid or methane sulfonic acid, advantageously be acetic acid.

The reaction of step (a) can advantageously be done in a solvent selected from toluene and isopropyl acetate, in particular isopropyl acetate.

The crystallization of step (b) is advantageously done from isopropyl acetate.

The reaction of step (a) and the subsequent crystalliaztion of step (b) can advantageously be done in isopropyl acetate as solvent.

The compound of formula (I), in particular in enantiomerically pure form, can be crystallized and subsequently isolated by filtration while the undesired enantiomer is removed as racemic mixture into the mother liquor.

The reaction of step (c) produces the compound of formula (I-c)

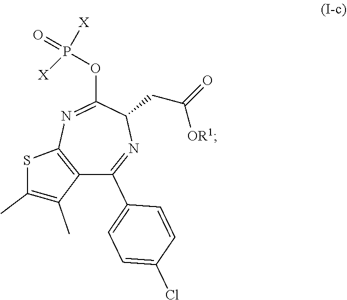

wherein X is —OEt, —OPh or

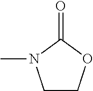

and R¹ is as defined above.

Step (d) produces the compound of formula (I-d) via the intermediate of formula (I-d′) or tautomer (I-d″)

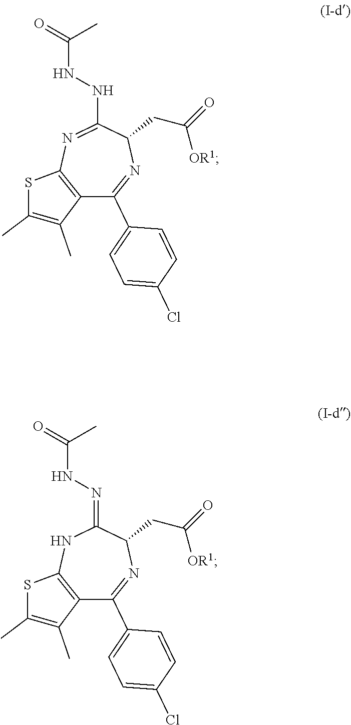

wherein R¹ is as defined above.

The compound of formula (I-d′) and/or (I-d″) are identifiable intermediates that cyclize to the compound of formula (I-d) at elevated temperatures, i.e. at temperature above room temperature.

Step (c) can be done at a temperature between e.g. −78° C. and room temperature with no racemization being observed.

In step (d), the reaction of the product of step (c) with acetyl hydrazide can advantageously be done at a temperature between −78° C. and 20° C.

The heating of step (d) above room temperature can advantageously be done at a temperature between 25° C. and 100° C. It forces the reaction to go to completion.

The product of step (c) can be used in step (d) as a crude product.

The product of step (d) can be used in step (e) as a crude product.

The compound of formula (I-e) can advantageously be obtained without isolating or purifying the intermediate products formed after steps (c) and (d).

The base of step (c) can advantageously be potassium tert.-pentoxide, potassium tert.-butoxide, sodium hydride, lithium tert.-pentoxide, lithium tert.-butoxide, sodium tert.-pentoxide or sodium tert.-butoxide more particularly potassium tert.-pentoxide.

In step (e), the deprotection of the carboxyl group of the compound of formula (I-d) consists in converting R¹ to a hydrogen atom.

Step (e) can be performed by reacting the product of step (d) with an acid or a base.

The acid of step (e) can advantageously be trifluoroacetic acid, in particular when R¹ tert.-butyl.

The base of step (e) can advantageously be sodium hydroxide, in particular in a solvent like methanol or methanol/water mixtures.

LiOH and Cs₂CO₃ can also be used in step (e).

Step (e) can for example advantageously be performed by reacting the product of step (d) with sodium hydroxide in a mixture of water and methanol.

The compound of formula (I-e) can for example be isolated after step (e) by crystallization from a mixture of isopropanol and n-heptane.

It was surprisingly found, in contradiction to what has been described in the art, that the reaction of step (c) can be done at temperatures above −10° C. with little to no concomittant racemization. Even at a temperature of 20° C., little or no racemization has been observed. Thus step (c) can be done without cooling below −10° C. or below room temperature, in particular without cooling below e.g. 20° C. Step (c) can thus be done at a temperature between around −78° C. and around 25° C., in particular between around 0° C. and around 20° C., in particular at room temperature.

The temperature of step (c) can advantageously be room temperature, for example around 20° C. or around 25° C.

No racemization has also been observed in steps (d) and (e).

The invention further relates to a process according to the invention wherein the compound of formula (II) is prepared by:
(f) the deprotection of the amino group R²—NH— of a compound of formula (III)

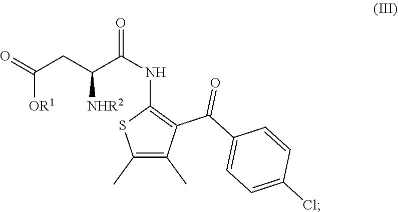

wherein R¹ is as defined above and R² is an amine protecting group.

R² is advantageously Fmoc.

If Fmoc is used as the amine protecting group, the deprotection of the amino group R²—NH— of the compound of formula (III) can be advantageously carried out by the reaction of a compound of formula (III) with a secondary amine, in particular piperazine, piperidine, morpholine or pyrrolidine, more particularly piperazine.

The invention further relates to a process according to the invention wherein the compound of formula (III) as defined above is prepared by:
(g) the reaction of a compound of formula (IV)

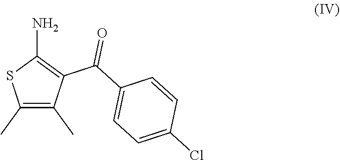 [SMILES: Cc1sc(N)c(C(=O)c2ccc(Cl)cc2)c1C]

with a compound of formula (V)

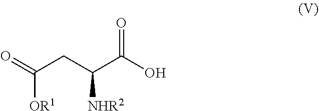

in the presence of a peptide coupling agent and optionally a base, wherein R¹ and R² are as defined above.

The compound of formula (III) is advantageously not isolated.

The peptide coupling agent of step (g) can be 1-[bis(dimethylamino)methylen]-5-chlorobenzotriazolium 3-oxide hexafluorophosphate (HCTU), 1-[bis(dimethylamino) methylen]-5-chlorobenzotriazolium 3-oxide hexafluorophosphate (HCTU) and hydroxybenzotriazole (HOBt), N,N,N′,N′-tetramethyl-O-(1H-benzotriazol-1-yl)uronium hexafluorophosphate (HBTU), N,N,N′,N′-tetramethyl-O-(1H-benzotriazol-1-yl)uronium hexafluorophosphate (HBTU) and hydroxybenzotriazole (HOBt), 1-[bis(dimethylamino)methylene]-1H-1,2,3-triazolo[4,5-b]pyridinium 3-oxide hexafluorophosphate (HATU), 1-[bis(dimethylamino)methylene]-1H-1,2,3-triazolo[4,5-b]pyridinium 3-oxide hexafluorophosphate (HATU) and hydroxybenzotriazole (HOBt), (benzotriazol-1-yloxy)tripyrrolidinophosphonium hexafluorophosphate (PyBOP), (benzotriazol-1-yloxy)tripyrrolidinophosphonium hexafluorophosphate (PyBOP) and hydroxybenzotriazole (HOBt) or propane phosphonic acid anhydride (T3P), in particular 1-[bis(dimethylamino)methylen]-5-chlorobenzotriazolium 3-oxide hexafluorophosphate (HCTU) or propane phosphonic acid anhydride (T3P).

The base of step (g) is advantageously diisopropylethylamine, N-methyl morpholine, triethylamine, lutidine or pyridine, in particular pyridine.

Examples of coupling conditions for step (g) can be HCTU/THF, HCTU/HOBt/THF, HBTU/HOBt/DCM, HBTU/HOBt/THF, HBTU/DCM, HATU/DMF, HATU/THF, HATU/HOBt/DMF, PyBOP/HOBt/DCM, PyBOP/DMF or T3P/pyridine/EA.

The reaction of step (g) can be done in a solvent selected from tetrahydrofuran, dichloromethane, dimethylformamide and ethyl acetate, in particular ethyl acetate.

In the reaction of step (g), the peptide coupling agent is more advantageously propane phosphonic acid anhydride (T3P), the base is advantageously pyridine and the solvent is advantageously ethyl acetate.

In the reaction of step (g), the peptide coupling agent is advantageously propane phosphonic acid anhydride (T3P) and the base is advantageously pyridine.

In the reaction of step (g), the peptide coupling agent is advantageously HCTU and the base is advantageously pyridine.

In the reaction of step (g), the peptide coupling agent is advantageously HCTU, the base is advantageously pyridine and the solvent is advantageously ethyl acetate.

It was surprisingly found that the coupling agents T3P or HCTU in step (g), and in particular T3P, provide the best enantiomeric purity for the compound of formula (IV).

The invention further relates to a process according to the invention wherein the compound of formula (IV) is prepared by the following steps:
(h) the reaction of 3-(4-chloro-phenyl)-3-oxo-propionitrile in the presence of butan-2-one, sulfur and a base to arrive at the compound of formula (IV);
(i) the formation of the oxalate salt of the compound of formula (IV); and
(j) the crystallization of the oxalate salt of the compound of formula (IV).

The base of step (h) is advantageously morpholine, diethylamine or 4-dimethylaminopyridine (DMAP), in particular 4-dimethylaminopyridine (DMAP).

DMAP can be used in amounts between 0.2 and 2 equivalents, more advantageously in sub-stoichiometric amounts, i.e. below 1 equivalent.

The oxalate salt of the compound of formula (IV) can be crystallized from a variety of solvents such as water, alcohols (eg . . . methanol, ethanol, isopropanol), esters (eg . . . methyl acetate, ethyl acetate, isopropyl acetate, n-butyl acetate, t-butyl acetate), acetonitrile, dichloromethane, chlorobenzene, in particular from acetonitrile.

It was surprisingly found that the formation of the oxalate salt of the compound of formula (IV) and its crystallization allows the removal of the undesired Gewald ethyl isomer (IV-a)

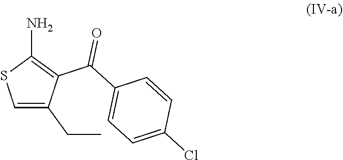 [SMILES: CCc1csc(N)c1C(=O)c1ccc(Cl)cc1]

that unavoidably also forms in the reaction. The process of the invention thus allows the preparation of the compound of formula (IV) in high purity.

The invention also relates to a process for purifying a compound of formula (IV) as defined above comprising forming the oxalate salt of the compound of formula (IV) and crystallizing said salt.

The above described crystallization conditions of the oxalate salt of the compound of formula (IV) can conveniently be used.

The invention further relates to a process for the manufacture of a compound of formula (III)

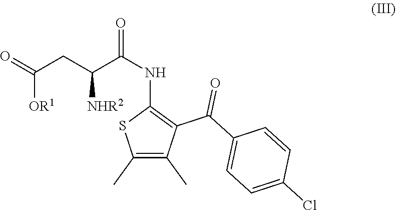

comprising:
(g) the reaction of a compound of formula (IV)

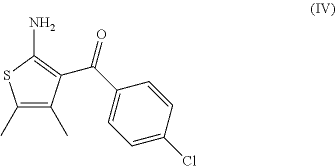 [SMILES: Cc1sc(N)c(C(=O)c2ccc(Cl)cc2)c1C]

with a compound of formula (V)

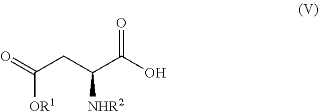

in the presence of a peptide coupling agent and optionally a base, wherein the peptide coupling agent is propane phosphonic acid anhydride (T3P) and wherein R¹ and R² are as defined above.

The base of step (g) is advantageously pyridine.

The solvent of step (g) is advantageously ethyl acetate.

The invention also relates to a process for the preparation of a compound of formula (IV)

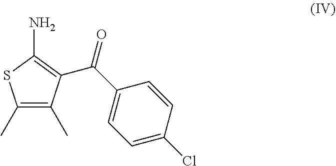 [SMILES: Cc1sc(N)c(C(=O)c2ccc(Cl)cc2)c1C]

comprising the following steps:
(h) the reaction of 3-(4-chloro-phenyl)-3-oxo-propionitrile in the presence of butan-2-one, sulfur and a base to arrive at the compound of formula (IV);
(i) the formation of the oxalate salt of the compound of formula (IV); and
(j) the crystallization of the oxalate salt of the compound of formula (IV).

The invention also relates to a process for the preparation of a compound of formula

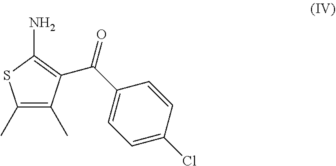 [SMILES: Cc1sc(N)c(C(=O)c2ccc(Cl)cc2)c1C]

comprising the following step:
(h) the reaction of 3-(4-chloro-phenyl)-3-oxo-propionitrile in the presence of butan-2-one, sulfur and DMAP to arrive at the compound of formula (IV).

The invention further relates in particular to a process for the manufacture of a compound of formula (I-e) as defined above comprising the following steps:
(c1) the reaction of a compound of formula (I) as defined above with diethyl chlorophosphate, diphenyl chlorophosphate or bis(2-oxo-3-oxazolidinyl)phosphinic chloride and a base at a temperature superior to −10° C.;
(c2) the reaction of a compound of formula (II) with bis(2-oxo-3-oxazolidinyl) phosphinic chloride and a base; or
(c3) the reaction of a compound of formula (II) having an enantiomeric ratio of at least 92:8 with diethyl chlorophosphate, diphenyl chlorophosphate or bis(2-oxo-3-oxazolidinyl)phosphinic chloride and a base;
(d) the reaction of the product of any one of steps (c1) to (c3) with acetyl hydrazide optionally followed by heating above room temperature to arrive at a compound of formula (I-d) as defined above; and
(e) the deprotection of the carboxyl group of the compound of formula (I-d) to arrive at the compound of formula (I-e) as defined above.

The invention also relates to a compound manufactured according to a process of the invention.

The process of the invention can be carried out according to the following scheme.

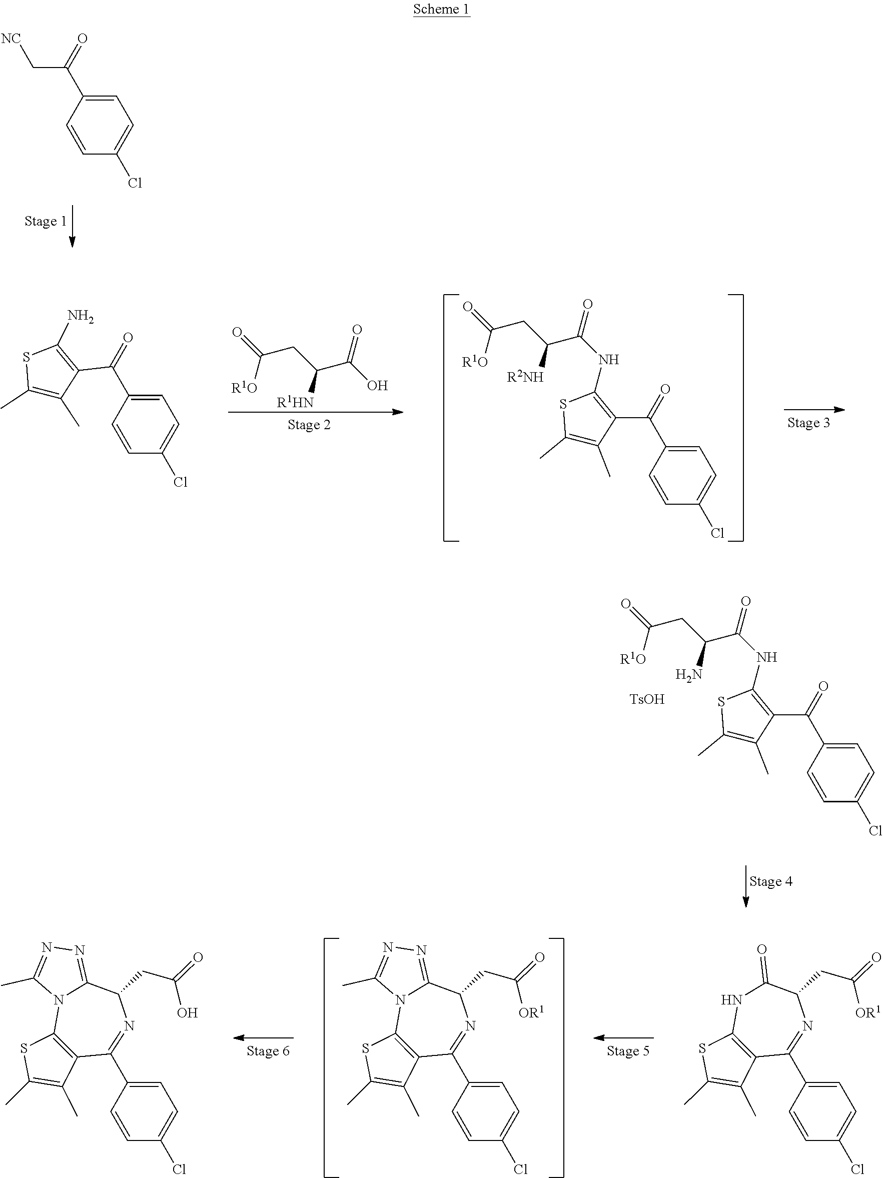

In scheme 1, R¹ and R² are as defined above.

The invention will now be illustrated by the following examples which have no limiting character.

### EXAMPLES

### Stage 1: Preparation of (2-amino-4,5-dimethyl-3-thienyl)-(4-chlorophenyl)methanone

Error! Objects cannot be created from editing field codes.

### Example 1.1: With Morpholine

This substance has been prepared using morpholine as the activating partner (eg. WO 2015/156601, WO 2015/131113, Angewandte Chemie, International Edition (2013), 52, 14060-14064, Journal of Biological Chemistry (2012), 287, 28840-28851, WO 2011/143660, Nature (2010), 468, 1067-1073 & U.S. Pat. No. 6,323,214) or with diethylamine (WO 2009/063301). In all reports, the product was purified by chromatography followed by recrystallization and no mention of the ethyl-isomer as side product has been recorded ever anywhere.

Purification Via Oxalate Salt

Crude aminothiophene (5.0 g, 19 mmol) and oxalic acid (1.7 g, 1 eq.) were taken up in methanol (50 ml). The light orange suspension was heated to reflux creating a dark red solution which then was cooled to ambient temperature. The brown suspension that formed was evaporated on a rotovap at 45° C./250-25 mb and the crude oxalate salt dried for 4 h at 45° C./25 mbar providing a yellow-orange crystalline solid (6.2 g, GC: 87% product, 13% ethyl isomer).

a) The oxalate salt (3.0 g) was taken up in acetonitrile (30 ml, 10× v/w) and the brown suspension was heated to reflux. The red solution produced was cooled and stirred at 25° C./1 h. A brown suspension arose which was filtered and the purified product was washed with dichloromethane (4 ml). The salt recovered was dried at 45° C./25 mb for 3 h and the filtrate evaporated.

Yield: 1.4 g yellow solid GC (area): 99% product, 1% ethyl isomer Filtrate: 1.6 g brown solid GC (area): 71% product, 25% ethyl isomer. The material from the filtrate was resuspended in acetonitrile (15 ml) and heated to reflux. After cooling to 25° C. the red solution was seeded with purified salt from above, cooled to 0-5° C. and stirred for 1 h. The precipitate was filtered, washed with dichloromethane (3 ml) and the isolated substance was dried at 45° C./25 mb for 3 h. The filtrate was evaporated.

Yield: 0.2 g yellow solid GC (area): 97% product, 3% ethyl isomer

Filtrate: 1.3 g brown resinGC (area): 58% product, 26% ethyl isomer

The first purified salt was partioned between ethyl acetate (25 ml) and 1N aqueous sodium hydroxide (25 ml). The organic phase was separated and washed with water (25 ml). The aqueous phase was extracted with ethyl acetate (25 ml). The combined organic extracts were dried over sodium sulphate, filtered & evaporated at 45° C./25 mb.

Yield: 1.2 g yellow solidGC (area): 99% product, 1% ethyl isomer (40-45% average recovery).

b) Alternatively, the oxalate salt (3.2 g) was taken up in acetonitrile (48 ml, 15× v/w) and the suspension was heated to reflux. The solution created was cooled, stirred at 25° C./1 h and for an additional 0.5 h at 0-5° C. The product was filtered and washed with dichloromethane (5 ml). The salt was dried for 3 h at 45° C./25 mb. The filtrate was evaporated.

Yield: 1.7 g yellow solidGC (area): ˜100% product, trace (<0.5%) ethyl isomer

Filtrate: 1.4 g brown solidGC (area): 67% product, 28% ethyl isomer

The first purified salt was partioned between ethyl acetate (25 ml) and 1N aqueous sodium hydroxide (25 ml). The organic phase was separated and washed with water (25 ml). The aqueous phase was extracted with ethyl acetate (25 ml), the combined organic extracts then dried over sodium sulphate, filtered and evaporated at 45° C./25 mb.

Yield: 1.5 g yellow solidGC (area): >99.5% product, <0.5% ethyl isomer (45-50% average recovery).

### Example 1.2: With DMAP

To 2-butanone (3.2 kg) in ethanol (48.0 kg) was added sequentially 4-chlorobenzoylacetonitrile (6.0 kg), 4-dimethylaminopyridine (1.0 kg) and sulfur (1.20 kg). The mixture was stirred under a nitrogen atmosphere at 25° C. for 3 h then at 75° C. for 18 h. Activated charcoal (0.3 kg) was added to the dark solution and after stirring for 0.5 h, the hot mixture was filtered, the residue washed with ethanol (5.0 kg) and the filtrate was poured into water (90.0 kg), maintained at 20-30° C., to precipitate the product. Stirring was continued for 2 h then at 5° C. after which the suspension was filtered. The filter cake was washed twice with a mixture of ethanol (5.0 kg) diluted with water (12.0 kg) and dried at 70° C. at 30 mb for 16 h. HPLC analysis indicated a purity of ˜75% with ˜14% ethyl-isomer and ˜1% starting nitrile.

The crude product was taken up in acetonitrile (28.8 kg), treated with oxalic acid (3.5 kg) and the mixture was stirred at 45° C. for 3 h. Upon completion of crystallization at 5° C./2 h, the oxalate salt was filtered, washed with cold (5° C.) acetonitrile (5.8 kg) and dried at 45° C. at 30 mb for 16 h.

The salt (6.7 kg) was released in a mixture of ethanol (10.9 kg) diluted with water (13.4 kg) by the addition of 5% aqueous potassium carbonate (56.8 kg). The slurry was stirred at 25° C. for 2 h and filtered. The product was washed with water (20.0 kg) then dried at 65° C. at 30 mb for 16 h. HPLC analysis indicated a purity of ˜93%, with ˜3% ethyl-isomer (50-55% average recovery).

### Stage 2: Preparation of Fmoc protected tert-butyl (3S)-3-amino-4-[[3-(4-chlorobenzoyl)-4,5-dimethyl-2-thienyl]amino]-4-oxo-butanoate

Error! Objects cannot be created from editing field codes.

### Example 2.1: with 2-(6-chloro-1H-benzotriazol-1-yl)-1,1,3,3-tetramethylaminium hexafluorophosphate (HCTU) pyridine as coupling aid

To (2-amino-4,5-dimethyl-3-thienyl)-(4-chlorophenyl)methanone (3.0 kg, from stage 1) and (S)-2-[(9H-fluoren-9-ylmethyl)-amino]-succinic acid 4-tert-butyl ester (6.9 kg) was added HCTU (9.3 kg) and pyridine (7.2 kg). The mixture was stirred at 25° C. under a nitrogen atmosphere for 18 h then diluted with isopropyl acetate (26.2 kg) and treated with 5% aqueous hydrochloric acid (38.0 kg).

The two phase solution (pH 3-4) was vigorously stirred at 25° C. for 0.5 h. The organic layer was separated and washed twice with 10% aqueous potassium carbonate solution (15.0 kg). The aqueous phases were back-extracted with isopropyl acetate (13.0 kg) and the combined organic extract was washed with 3% aqueous sodium chloride solution (15.0 kg). After concentrating the organic extract under reduced pressure at 45° C. to 2-3 vol., more isopropyl acetate (8.8 kg) was added and the process repeated to azeotropically dry the solution. The concentrate was diluted with isopropyl acetate (6.6 kg) and the solution used directly in the next step.

A sample evaporated to dryness indicated an average yield of ˜85% and 97% ee (over several runs).

### Example 2.2: with 2,4,6-tripropyl-1,3,5,2,4,6-trioxatriphosphorinane-2,4,6-trioxide (T3P) pyridine as coupling aid

To (2-amino-4,5-dimethyl-3-thienyl)-(4-chlorophenyl)methanone (30.0 g, from stage 1) and (S)-2-[(9H-fluoren-9-ylmethyl)-amino]-succinic acid 4-tert-butyl ester (69.7 g) in ethyl acetate (60 ml) was added 50% T3P in ethyl acetate (143.6 g) followed by pyridine (35.8 g). The mixture was stirred at 25° C. under a nitrogen atmosphere for 18 h then diluted with isopropyl acetate (300 ml) and treated with 2.5N aqueous hydrochloric acid (200 ml).

After vigorously stirring the two phase solution (pH 2-3) at 25° C. for 0.5 h, the organic layer was separated and washed 3% aqueous sodium chloride solution (150 ml). The combined aqueous phases were back-extracted with isopropyl acetate (120 ml) and the combined organic extract was concentrated under reduced pressure at 40° C. to 2-3 vol.

More isopropyl acetate (180 ml) was added and the process repeated to azeotropically dry the solution. The concentrate was diluted with isopropyl acetate (180 ml) and the solution used directly in the next step.

A sample evaporated to dryness indicated an average yield of ˜90% and 99% ee (over several runs).

### Example 2.3

Several reaction conditions and reagents have been tested. The results are given in the following tables.

TABLE 1

| | | Ratio of Fmoc- | | | | |
|---|---|---|---|---|---|---|
| | | AA:coupling | Reaction | Product % | Educt % | |
| Batch | Coupling conditions | reagent:base:Educt | time | (HPLC) | (HPLC) | S:R |
| 1 | HCTU/THF | 2:2:4:1 | 20 h | 68.40 | 0.96 | 74.4:25.6 |
| 2 | HCTU/HOBt/THF | 2:2:4:1 | 18 h | 77.60 | 20.56 | 75.9:24.1 |
| 3 | HBTU/HOBt/DCM | 1:1:3.5:1 | 17 h | 40.13 | 32.09 | 82.0:18.0 |
| | | 2:2:4:1 | 22 h | 66.76 | 19.57 | 87.4:12.6 |
| 4 | HBTU/HOBt/DCM | 2:2:4:1 | 18 h | 69.38 | 22.05 | 67.9:32.1 |
| | | | 47 h | 81.18 | 11.14 | 73.0:27.0 |
| 5 | HBTU/HOBt/THF | 2:2:4:1 | 18 h | 25.19 | 57.37 | 61.4:38.6 |
| 6 | HBTU/HOBt/THF | 2:2:4:1 | 18 h | 33.29 | 58.89 | 60.8:39.2 |
| 7 | HBTU/DCM | 2:2:4:1 | 18 h | 62.58 | 1.29 | 67.2:32.8 |

TABLE 2

| | | Ratio of | | | | |
|---|---|---|---|---|---|---|
| | | Fmoc-AA:coupling | Reaction | Product | Educt % | |
| Batch | Coupling conditions | reagent:base:Educt | time | % (HPLC) | (HPLC) | S:R |
| 8 | HATU/DMF | 2:2:4.4:1 | 22 h | 38.81 | 7.79 | 80.4:19.6 |
| 9 | HATU/THF | 2:2:4.4:1 | 22 h | 35.92 | 11.97 | 80.0:20.0 |
| 10 | HATU/HOBt/DMF | 2:2:4:1 | 18 h | 46.52 | 42.30 | 82.8:17.2 |
| 11 | PyBOP/HOBt/DCM | 2:2:4:1 | 47 h | 53.41 | 10.62 | 69.5:30.5 |
| 12 | PyBOP/DMF | 2:2:5.5:1 | 46 h | 30.59 | 11.20 | 78.5:21.5 |

TABLE 3

| | | Ratio of Fmoc- | | | | |
|---|---|---|---|---|---|---|
| | | AA:coupling | reaction | Product | Educt % | |
| Batch | Coupling conditions | reagent:base:Educt | time | % (HPLC) | (HPLC) | S:R |
| 13 | T3P/Pyridine/EA | 2:2:4:1 | 20 h | 81.36 | 12.71 | 99.3:0.7 |
| | | | 65 h | 86.45 | 7.56 | 99.3:0.7 |
| 14 | T3P/Pyridine/EA | 2:2:4:1 | 18 h | 78.06 | 18.06 | 99.5:0.5 |
| | | | 24 h | 78.86 | 16.67 | 99.5:0.5 |
| | | | 42 h | 85.43 | 8.92 | 99.6:0.4 |
| | | | 66 h | 89.15 | 6.50 | 99.6:0.4 |

### Stage 3: Preparation of the tosylate salt of tert-butyl (3S)-3-amino-4-[[3-(4-chlorobenzoyl)-4,5-dimethyl-2-thienyl]amino]-4-oxo-butanoate

Error! Objects cannot be created from editing field codes.

### Example 3

The solution from stage 2, (prepared as described in example 2.1) was diluted with additional isopropyl acetate (16.5 kg), piperazine (1.65 kg) was added and the mixture was stirred at 25° C. for 16 h. The slurry was filtered through celite (1.6 kg) with the aid of isopropyl acetate (2×9.5 kg), the filtrate treated with 5% aqueous hydrochloric acid (19.0 kg) and the two phase solution (pH 3-4) was vigorously stirred at 25° C. for 0.5 h.

The organic layer was separated then washed with 10% aqueous potassium carbonate solution (31.6 kg) and 3% aqueous sodium chloride solution (31.6 kg). The aqueous phases were sequentially back-extracted with isopropyl acetate (6.4 kg) and to the combined organic extract after separating residual water was added a total of p-toluenesulphonic acid monohydrate (1.82 kg) and t-butylmethylether (35.4 kg) in three portions both over 0.5 h.

The suspension was stirred at 25° C. for 6 h and filtered. The residue was washed with t-butylmethylether (4×6.3 kg) and dried at 60° C. at 30 mb for 16 h.

An average yield of ˜80% and 98% ee was attained over several runs.

### Stage 4: Preparation of tert-butyl 2-[(3S)-5-(4-chlorophenyl)-6,7-dimethyl-2-oxo-1,3-dihydrothieno[2,3-e][1,4]diazepin-3-yl]acetate

Error! Objects cannot be created from editing field codes.

### Example 4.1: With Substrate of High Enantiomeric Purity

The tosylate salt (2.0 kg) from stage 3 (98% ee) was taken up in isopropyl acetate (10.6 kg) and treated with 10% aqueous potassium carbonate solution (13.1 kg). The mixture was stirred at 25° C. for 2 h then filtered. The residue was rinsed with isopropyl acetate (2×2.0 kg) and filtrate was washed with water (2.7 kg). The aqueous phases were back-extracted sequentially with isopropyl acetate (4.7 kg) and to the combined organic extract was added acetic acid (0.2 kg).

The solution was heated at 90° C. for 3 h with the azeotropic removal of water. After cooling to 70° C., the reaction mixture was washed with preheated (70° C.) 10% aqueous potassium carbonate solution (2×4 kg) and water (2.7 kg). The aqueous phases were back-extracted successively with isopropyl acetate (4.0 kg) and the combined organic extract dried by azeotropic distillation at 90° C. The hot solution was filtered and the residue washed with isopropyl acetate (2.0 kg). Distillation at 90° C. was continued until ca. 3 vol were reached and crystallization was completed thereafter at 20° C. for 4 h. The product was filtered, washed with isopropyl acetate (2.0 kg) and dried at 60° C. at 30 mb for 10 h.

An average yield of ˜70% and 100% ee was acquired over several runs.

### Example 4.2: With Substrate of Low Enantiomeric Purity

To the tosylate salt (80.0 g) from a stage 3 run (displaying an enantiomeric ratio of 73:27) suspended in isopropyl acetate (480 ml), was added 10% aqueous potassium carbonate solution (480 ml) and the mixture was stirred at 25° C. for 2 h. The organic phase was separated, washed with water (100 ml) and treated with acetic acid (7.9 g).

The solution was heated at 90° C. for 3 h with the azeotropic removal of water. Additional isopropyl acetate (320 ml) was added, the solution cooled to 40° C. and washed with warm 10% aqueous potassium carbonate solution (2×200 ml) and water (100 ml). The solvent was dried by azeotropic distillation at 90° C. After cooling to 20° C., crystallization was effected over 4h. The product was filtered, washed in portions with isopropyl acetate (100 ml) and dried at 60° C. at 30 mb for 10 h; a yield ˜40% and purity of 100% ee was obtained.

### Stage 5: Preparation of t-butyl [(S)-4-(4-chloro-phenyl)-2,3,9-trimethyl-6H-1-thia-5,7,8,9a-tetraaza-cyclopenta[e]azulen-6-yl]-acetate

Error! Objects cannot be created from editing field codes.

### Example 5.1: With Potassium Amylate as Base and Diphenyl Chlorophosphate for Activation

A solution of the product from stage 4 (20.0 g) in tetrahydrofuran (320 ml) was cooled to −40° C. and treated dropwise over 1 h with a 25% toluene solution of potassium amylate (27.3 g). After stirring at −40° C. for 1 h, a solution of diphenyl chlorophosphate (16.8 g) in tetrahydrofuran was added over 0.3 h. The reaction mixture was warmed to −10° C. over 1.5 h and stirred at this temperature for 0.5 h.

A suspension of acetyl hydrazide (5.1 g) in toluene (30 ml) was added with the aid of additional toluene (30 ml) and the mixture was allowed to warm over 0.5 h to 20° C.

Stirring was continued for 1 h, more toluene (200 ml) added and the reaction mixture was heated at 80° C. for 1 h.

Solvent was removed under reduced pressure to a residual volume of ca. 400 ml, water (80 ml) was added and the two phase mixture was stirred at 20° C. for 0.3h. The organic layer was separated and washed with 0.1N aqueous sulphuric acid (80 ml), 5% aqueous sodium carbonate (80 ml) and water (80 ml) then evaporated under reduced pressure, yielding crude stage 5 product (˜25 g) which was used directly in the subsequent step.

The deprotection can also be conducted with potassium tert.-butoxide and/or at temperatures of up to 20° C. with essentially no depreciation on yield or enantiomeric purity.

### Example 5.2: With Sodium Hydride as Base and Bis(2-Oxo-3-Oxazolidinyl)Phosphinic Chloride for Activation

To a suspension of sodium hydride (60% in oil, 30 mg, 0.75 mmol) in dry tetrahydrofuran (1 ml) cooled to 0-5° C. was added a solution of the product from stage 4 (209 mg, 0.5 mmol) in dry tetrahydrofuran (1.5 ml) over 5 min. The yellow solution was stirred for 5 min and a solution of bis(2-oxo-3-oxazolidinyl)-phosphinic chloride (197 mg, 0.75 mmol) was added in one portion. The yellow suspension formed was stirred at 0-5° C. for 2h.

HPLC (area): 93% iminophosphate intermediate and 1% starting material.

Acetyl hydrazide (82 mg, 1 mmol) was added in one portion and the light brown suspension created was stirred 1.25h at 20° C.

HPLC (area): 0% iminophosphate intermediate, 76% iminohydrazide intermediate, 4% triazole product and 2% starting material. The reaction mixture was heated at 65° C. for 1h to complete the ring closure step.

The suspension was partioned between ethyl acetate (10 ml) and water (10 ml). The organic layer was separated and washed with water (10 ml). The aqueous phases were back extracted with ethyl acetate (10 ml) and the combined organic extracts were dried over sodium sulphate, filtered and evaporated.

Yield: 230 mg light brown syrup (˜100%). HPLC (area %) analysis indicated a purity of ˜93%, with 2% of remaining starting material.

### Stage 6: Preparation of [(S)-4-(4-chloro-phenyl)-2,3,9-trimethyl-6H-1-thia-5,7,8,9a-tetraaza-cyclopenta[e]azulen-6-yl]-acetic acid

Error! Objects cannot be created from editing field codes.

### Example 6.1: With Trifluoroacetic Acid

The product from stage 5 (24.6 g; prepared as described in Example 5.1; 24.6 g) was dissolved in trifluoroacetic acid (80 ml) and the solution was stirred at 20° C. for 2 h. The solvent was removed under reduced pressure and the residue was taken up in toluene (200 ml). Excess trifluoroacetic acid was eliminated through concentration under reduced pressure.

The crude product, as the trifluoroacetate salt, was taken up in water (200 ml) and treated with 28% aqueous sodium hydroxide (35 g) rendering a pH ca. 10. t-Butyl methyl ether (200 ml) was added and the pH of the aqueous phase was adjusted to pH 7.3-7.5 with 5% aqueous sulphuric acid (50 g). After vigorously stirring the two phase mixture for 0.3 h, the organic layer was separated and to the aqueous phase containing the product was added t-butyl methyl ether (200 ml). The pH of the aqueous phase was adjusted further to pH 6.4-6.6 with 5% aqueous sulphuric acid (10 g) and the mixture was stirred for 0.3 h. The organic layer containing residual stage 4 deprotected product acid was separated and the aqueous phase containing the product was extracted with t-butyl methyl ether (ca. six times) until the level of contaminant acid in the aqueous layer was <0.5 area-% by HPLC. Dichloromethane (160 ml) was added to the aqueous phase, the pH lowered to 5.8-6.0 with % aqueous sulphuric acid (25 ml) and the mixture stirred for 0.3 h. The aqueous phase was back extracted with dichloromethane (100 ml) and the combined organic extracts were evaporated under reduced pressure.

The product was suspended in isopropanol (60 ml), residual dichloromethane removed by concentration at 40° C./40 mb and the residue resuspended in isopropanol (60 ml). The mixture was heated to 65° C., stirred until a clear, orange solution was obtained, then allowed to cool to 20° C. whereupon the product partially precipitated. The suspension was stirred for 1 h, diluted over 1h with n-heptane (120 ml) and stirred for 2 h at 20° C. The product was filtered, washed with 10% isopropanol in heptane (50 ml) and dried at 60° C./10 mb for 16 h, furnishing stage 6 product (10.4 g, ca. 55% over two steps, ee 100%) as a pale yellow powder.

### Example 6.2: With Aqueous Sodium Hydroxide

The product from stage 5 (21.8 g) was dissolved in methanol (65 ml) at 40° C. and treated with 28% aqueous sodium hydroxide (10.4 ml). The solution was diluted with water (7 ml) and stirred at 40° C. for 4 h. The reaction mixture was cooled to 20° C. then partitioned between water (175 ml) and t-butyl methyl ether (220 ml). The pH of the aqueous phase was adjusted to ca. 10 with sulphuric acid (1.5 ml) diluted in water (55 ml). After stirring for 0.2 h, the pH of the separated aqueous layer was lowered to ca. 7.5 with sulphuric acid (1.5 ml) diluted in water (55 ml) and the phase extracted with t-butyl methyl ether (220 ml). The pH was further adjusted to 6.5 with sulphuric acid (0.1 ml) diluted in water (20 ml) and the extraction with t-butyl methyl ether (220 ml) repeated. Maintaining the pH at 6.5 by the same means, the extraction with t-butyl methyl ether was performed two more times. Finally the pH was set at 5.9 with sulphuric acid (0.8 ml) diluted in water (5 ml) and the product extracted into dichloromethane (220 ml). The extraction of the separated aqueous phase with dichloromethane was repeated whilst retaining the pH at 5.9 through judicious addition of aqueous sulphuric acid and the combined organic extracts then were evaporated under reduced pressure.

The product was taken up in isopropanol (400 ml), filtered, residual dichloromethane removed by concentration at 50° C./60 mb and the residue redissolved in isopropanol (33 ml). n-Heptane (15 ml) was added dropwise, the mixture seeded and stirring continued for 16 h at 20° C. Additional n-heptane (40 ml) was added over 0.5 h and after stirring at 20° C. for a further 5 h, the suspension was filtered. The residue was washed with 65% isopropanol in heptane (40 ml) and heptane (20 ml) then dried at 60° C./10 mb for 16 h, delivering stage 6 product (9.3 g, ca. 50% over two steps, ee 100%) as a pale yellow powder.

1. A process for the manufacture of a compound of formula (I) 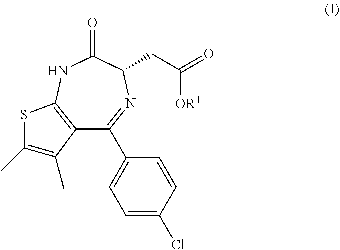
comprising the following steps:
(a) the reaction of a compound of formula (II) 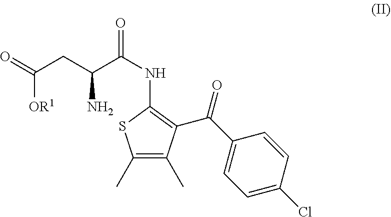
having an enantiomeric ratio of at least 70:30 with an acid to arrive at the compound of formula (I); and
(b) the crystallization of the compound of formula (I) obtained in step (a) from isopropyl acetate;
wherein R¹ is alkyl.

2. A process according to claim 1, wherein the reaction of step (a) is done in toluene or isopropyl acetate, in particular isopropyl acetate.

3. A process according to claim 1 or 2, wherein water that is produced during the reaction of step (a) is removed from the reaction mixture.

4. A process according to any one of claims 1 to 3, wherein the acid of step (a) is acetic acid, formic acid or methane sulfonic acid, particularly acetic acid.

5. A process according to any one of claims 1 to 4, further comprising:
(c) the reaction of a compound of formula (I) as defined in claim 1 with diethyl chlorophosphate, diphenyl chlorophosphate or bis(2-oxo-3-oxazolidinyl)phosphinic chloride and a base;
(d) the reaction of the product of step (c) with acetyl hydrazide followed by heating above room temperature to arrive at the compound of formula (I-d) 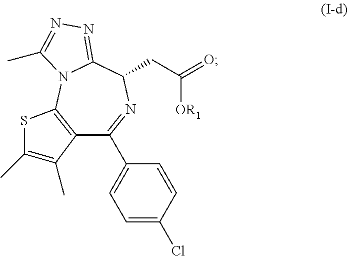
and
(e) the deprotection of the carboxyl group of the compound of formula (I-d) to arrive at the compound of formula (I-e) 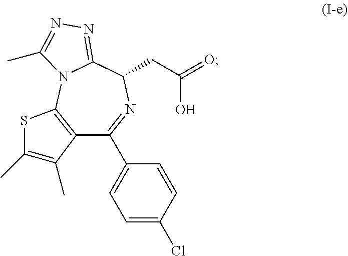
wherein R¹ is as defined in claim 1.

6. A process according to any one of claims 1 to 5, wherein the compound of formula (II) as defined in claim 1 is prepared by:
(f) the deprotection of the amino group R²—NH— of a compound of formula (III) 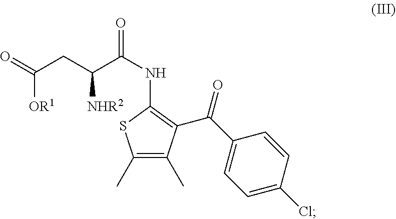
wherein R¹ is as defined in claim 1 and R² is an amine protecting group.

7. A process according to claim 6, wherein the compound of formula (III) as defined in claim 6 is prepared by:
(g) the reaction of a compound of formula (IV) or a salt thereof 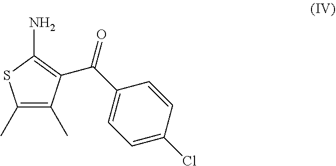 [SMILES: Cc1sc(N)c(C(=O)c2ccc(Cl)cc2)c1C]
with a compound of formula (V) 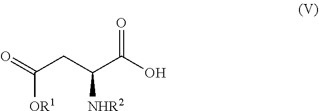
in the presence of a peptide coupling agent and optionally a base, wherein R¹ is as defined in claim 1 and R² is as defined in claim 6.

8. A process according to claim 7, wherein the peptide coupling agent is 1-[bis(dimethylamino)methylen]-5-chlorobenzotriazolium 3-oxide hexafluorophosphate (HCTU), 1-[bis(dimethylamino)methylen]-5-chlorobenzotriazolium 3-oxide hexafluorophosphate (HCTU) and hydroxybenzotriazole (HOBt), N,N,N′,N′-tetramethyl-O-(1H-benzotriazol-1-yl)uronium hexafluorophosphate (HBTU), N,N,N′,N′-tetramethyl-O-(1H-benzotriazol-1-yl)uronium hexafluorophosphate (HBTU) and hydroxybenzotriazole (HOBt), 1-[bis(dimethylamino)methylene]-1H-1,2,3-triazolo[4,5-b]pyridinium 3-oxide hexafluorophosphate (HATU), 1-[bis(dimethylamino)methylene]-1H-1,2,3-triazolo[4,5-b]pyridinium 3-oxide hexafluorophosphate (HATU) and hydroxybenzotriazole (HOBt), (benzotriazol-1-yloxy)tripyrrolidinophosphonium hexafluorophosphate (PyBOP), (benzotriazol-1-yloxy)tripyrrolidinophosphonium hexafluorophosphate (PyBOP) and hydroxybenzotriazole (HOBt) or propane phosphonic acid anhydride (T3P), in particular propane phosphonic acid anhydride (T3P).

9. A process according to claim 7 or 8, wherein the base of step (g) is diisopropylethylamine, N-methyl morpholine, triethylamine, lutidine or pyridine, in particular pyridine.

10. A process according to any one of claims 7 to 9, wherein the compound of formula (IV) as defined in claim 7 is prepared by the following steps:
(h) the reaction of 3-(4-chloro-phenyl)-3-oxo-propionitrile in the presence of butan-2-one, sulfur and a base to arrive at the compound of formula (IV);
(i) the formation of the oxalate salt of the compound of formula (IV); and
(j) the crystallization of the oxalate salt of the compound of formula (IV).

11. A process according to claim 10, wherein the base is morpholine, diethylamine or 4-dimethylaminopyridine (DMAP), in particular 4-dimethylaminopyridine (DMAP).

12. A process according to claim 10 or 11, wherein the oxalate salt of the compound of formula (IV) as defined in claim 7 is crystallized from water, alcohols, in particular methanol, ethanol or isopropanol, esters, in particular methyl acetate, ethyl acetate, isopropyl acetate, n-butyl acetate or t-butyl acetate, acetonitrile, dichloromethane or chlorobenzene, in particular acetonitrile.

13. A process for the manufacture of a compound of formula (III) as defined in claim 6 comprising:
(g) the reaction of a compound of formula (IV) 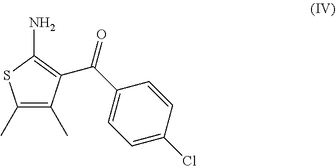 [SMILES: Cc1sc(N)c(C(=O)c2ccc(Cl)cc2)c1C]
with a compound of formula (V) 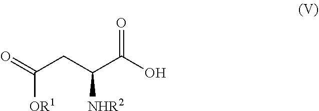
in the presence of a peptide coupling agent and optionally a base, wherein the peptide coupling agent is propane phosphonic acid anhydride (T3P) and wherein R¹ is as defined in claim 1 and R² is as defined in claim 6.

14. A process for the preparation of a compound of formula (IV) 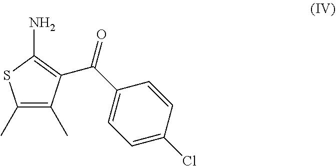 [SMILES: Cc1sc(N)c(C(=O)c2ccc(Cl)cc2)c1C]
comprising the following steps:
(h) the reaction of 3-(4-chloro-phenyl)-3-oxo-propionitrile in the presence of butan-2-one, sulfur and a base to arrive at the compound of formula (IV);
(i) the formation of the oxalate salt of the compound of formula (IV); and
(j) the crystallization of the oxalate salt of the compound of formula (IV).

15. A process for the preparation of a compound of formula (IV) 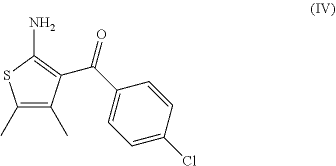 [SMILES: Cc1sc(N)c(C(=O)c2ccc(Cl)cc2)c1C]
comprising the following step:
(h) the reaction of 3-(4-chloro-phenyl)-3-oxo-propionitrile in the presence of butan-2-one, sulfur and DMAP.

16. A process for purifying a compound of formula (IV) as defined in claim 7, comprising forming the oxalate salt of the compound of formula (V) and crystallizing said salt.

17. A process for purifying a compound of formula (I) as defined in claim 1 comprising the crystallization of the compound of formula (I) having an enantiomeric ratio of at least 70:30 from isopropyl acetate.

18. A process according to any one of claims 1 to 17, wherein R¹ is tert.-butyl.

19. A process according to any one of claims 6 to 18, wherein R² is Fmoc.

20. A compound manufactured according to a process of any one of claims 1 to 19.

21. The invention as hereinbefore described
